(19) United States
(12) Patent Application Publication
(10) Pub. No.: US 2025/0295915 A1
(43) Pub. Date: Sep. 25, 2025

(54) ELECTRICAL STIMULATION GENERATING DEVICE FOR IMPROVING SLEEP DISORDERS AND THE METHOD THEREOF

(71) Applicant: Taiwan Resonant Waves Research Corp., Taipei City (TW)

(72) Inventor: Ying-Chuan CHIU, Taipei City (TW)

(21) Appl. No.: 18/894,735

(22) Filed: Sep. 24, 2024

(30) Foreign Application Priority Data
Mar. 21, 2024 (TW) 113110436

(51) Int. Cl. A61N 1/36; A61N 1/04

(52) U.S. Cl. CPC A61N 1/36034; A61N 1/048; A61N 1/36025

(57) ABSTRACT

The present invention relates to an electrical stimulation generating device for improving sleep disorders and the method thereof. The electrical stimulation generating device is used to generate a current, and the current has an electrical stimulation pulse. Finally, the current with the electrical stimulation pulse is transmitted to the skin of the individual, which can improve sleep disorders.

### CROSS-REFERENCE TO RELATED APPLICATION

This application claims the benefit of Taiwan Patent Application Serial No. 113110436 filed on Mar. 21, 2024. The entirety of each Application is incorporated herein by reference.

### BACKGROUND OF THE INVENTION

### 1. Field of the Invention

The present invention relates to an electrical stimulation generating device for improving sleep disorders and the method thereof, particularly an electrical stimulation generating device and a method for generating low-frequency electrical stimulation signals and transmitting them to user's body for improving sleep disorders.

### 2. Description of Related Art

The primary function of sleep is to repair the body's physical exertion during the day, restore the immune system, and organize emotional behavior and cognitive memory. Abnormal sleep-related behaviors, such as difficulty falling asleep or excessive sleep duration, are referred to as sleep disorders. Among these, insomnia is one of the most common types. Sleep disorders can lead to negative consequences, including physical fatigue, immune system dysfunction, hormonal imbalances, and psychological or mental health issues. Currently, drug treatment and behavioral therapy are the most commonly used methods. While drug treatment can quickly induce sleep, it may lead to drug addiction as a side effect. Therefore, there is a significant need for a non-drug, gentle, and non-invasive treatment technology.

Furthermore, transcutaneous electrical nerve stimulators (TENS), also known as low-frequency therapy devices, are a well-known electrical stimulation generation technology. It mainly uses an electrical stimulation generator to produce a current signal and transmits the signal to the human body for stimulation through the electrode patches attached to the skin. These devices are commonly used for relieving muscle soreness and pain or for muscle training through electrical stimulation signals. However, commercially available low-frequency devices use biphasic electrical pulses, generating pulse signals by alternating positive and negative phases. Some devices use intermediate-frequency interference wave signals created by superimposing two frequencies. The output voltages generated by the aforementioned devices are all high volts (approximately 100V). At present, low-frequency devices are rarely used to improve or treat sleep disorders. For instance, using devices with higher frequencies or voltages is not suitable for individuals with sleep disorders. Prolonged use of such devices may lead to muscle fatigue, which can further cause involuntary muscle tremors.

In light of the above, it is necessary to address the issues present in existing electrical stimulation technology, such as unstable output voltage, the inability to sustain higher frequencies over extended periods, and concerns about safety of applications to individual. The present invention provides an electrical stimulation device that can generate and transmit low-frequency electrical stimulation signals to the individual through the skin via the electrode structure. This device can effectively improve user's sleep disorders (such as insomnia) and its low-frequency electrical stimulation signals will not cause muscle fatigue typically associated with common electrical stimulation.

### SUMMARY OF THE INVENTION

An object of the present invention is to provide an electrical stimulation generating device for improving sleep disorders and a method capable of generating electrical stimulation signals, thereby applying electrical stimulation pulses to the human body for improving sleep disorders. In addition, the electrical stimulation generating device of the present invention generates current that is transmitted to the human body through the electrode structure. The output voltage is suitable for human use, operates within a safe range, and minimizes risk. Compared to devices available on the market, the electrical stimulation signals produced by the present invention are gentler and imperceptible. Moreover, this electrical stimulation generating device is capable of programmable settings of different output frequencies, enabling users to select and operate according to their needs. It is a non-invasive and conveniently portable electrical stimulation generating device. Thus, the electrical stimulation signals generated by using this device are effective in improving insomnia.

The present invention provides an electrical stimulation generating device for improving sleep disorders, comprising: a host machine, a signal generator, at least one signal transmission module, and at least one electrode structure. The signal generator is disposed within the host machine for generating a current. The signal transmission module has one end electrically connected to the signal generator. The electrode structure is electrically connected to the other end of the signal transmission module and has a contact surface for contacting the skin of an individual and transmitting the current to the individual. Wherein the current generated by the signal generator has an electrical stimulation pulse with a frequency of 1 to 30 Hz (hertz), and includes a gradually increasing frequency group, a constant frequency group, or a gradually decreasing frequency group. The gradually increasing frequency group, the constant frequency group, and the gradually decreasing frequency group are each composed of a plurality of single-phase pulse groups, and a duty cycle of the single-phase pulse group is more than 60%.

In one embodiment, the frequency of the gradually increasing frequency group and the gradually decreasing frequency group range from 5 to 30 Hz, and the frequency of the constant frequency group ranges from 4 to 8 Hz.

In one embodiment, the output time of the gradually increasing frequency group is between 350 and 470 seconds, the output time of the gradually decreasing frequency group is between 170 and 250 seconds, and the output time of the constant frequency group is between 1750 and 1850 seconds.

In one embodiment, the gradually increasing frequency group sequentially includes a first gradually increasing frequency, a second gradually increasing frequency, and a third gradually increasing frequency, the constant frequency group sequentially includes a first constant frequency, a second constant frequency, a third constant frequency, a fourth constant frequency, and a fifth constant frequency, and the gradually decreasing frequency group sequentially includes a first gradually decreasing frequency, a second gradually decreasing frequency, and a third gradually decreasing frequency.

In one embodiment, each of the first gradually increasing frequency to the third gradually increasing frequency, the first constant frequency to the fifth constant frequency, and the first gradually decreasing frequency to the third gradually decreasing frequency have their respective frequency ranges and output times.

In one embodiment, the duty cycles of the single-phase pulse groups are 70% or 100%.

In one embodiment, the single-phase pulse groups are composed of a plurality of single-phase pulses with the duty cycle of 40% to 60%.

In one embodiment, the waveform of the electrical stimulation pulse is a square wave, a sine wave, a triangle wave, or a sawtooth wave.

In one embodiment, a potential difference of the current is less than 10 Vpp.

In one embodiment, the signal transmission module is a signal transmission wire or a combination of the signal transmission wire and a magnetic clasp.

In one embodiment, the electrode structure contacts the abdominal skin of the individual.

The present invention further provides a method for generating an electrical stimulation pulse on the electrical stimulation generating device, wherein the electrical stimulation pulse generated by the method is sequentially transmitted from the signal generator through the electrode structure to the individual. After the electrical stimulation pulse is applied to the individual, it can effectively improve the sleep disorders compared to the effects that previous technologies could not achieve.

To achieve the above purpose, the present invention provides an electrical stimulation generating device capable of improving sleep disorders and a method for generating the electrical stimulation pulse.

After referring to the drawings and the implementation methods described later, those with ordinary knowledge in this field can understand other objectives of the present invention, as well as the technical means and implementation modes of the present invention.

### BRIEF DESCRIPTION OF THE DRAWINGS

FIG. 1 is a schematic view of an embodiment of the electrical stimulation generating device according to the present invention;

FIG. 2 is a schematic view of the system block inside the electrical stimulation generating device according to the present invention;

FIG. 3 is a schematic view of the electrical stimulation pulse in one embodiment according to the present invention;

FIG. 4 is a schematic view of the electrical stimulation pulse displayed on an oscilloscope in one embodiment according to the present invention;

FIG. 5 is a schematic view of the potential difference generated by the electrical stimulation pulse in one embodiment according to the present invention;

FIG. 6 is a schematic view of a single-phase pulse group composed of single-phase pulse waves and the duty cycle of the single-phase pulse group in one embodiment according to the present invention;

FIG. 7 is a schematic view of the electrical stimulation pulse used to improve sleep disorders in one embodiment according to the present invention;

FIG. 8a is a quantified analysis result diagram of the insomnia severity and the time in bed before and after using the electrical stimulation generating device in one embodiment according to the present invention;

FIG. 8b is a quantified analysis result diagram of the insomnia severity index before and after using the electrical stimulation generating device in one embodiment according to the present invention;

FIG. 8c is an evaluation score result diagram of the insomnia severity index before and after using the electrical stimulation generating device in one embodiment according to the present invention;

FIG. 9 is a schematic view of another embodiment of the electrical stimulation generating device according to the present invention;

FIG. 10 is a schematic view of another embodiment of the electrical stimulation generating device according to the present invention;

FIG. 11 is an exploded view of another embodiment of the electrical stimulation generating device according to the present invention; and

FIG. 12 is an exploded view of another embodiment of the electrical stimulation generating device according to the present invention.

### DETAILED DESCRIPTION OF THE PREFERRED EMBODIMENT

Reference will now be made in detail to the present embodiments of the invention, examples of which are illustrated in the accompanying drawings, and are not intended to limit the present invention, applications, or implementations described in these embodiments. Wherever possible, the same reference numbers are used in the drawings and the description to refer to the same or like parts. It should be noticed that, in the following embodiments and the attached drawings, elements unrelated to the present invention are omitted from the depiction; and dimensional relationships among individual elements in the drawings are provided only for ease of understanding, but not to limit the actual scale.

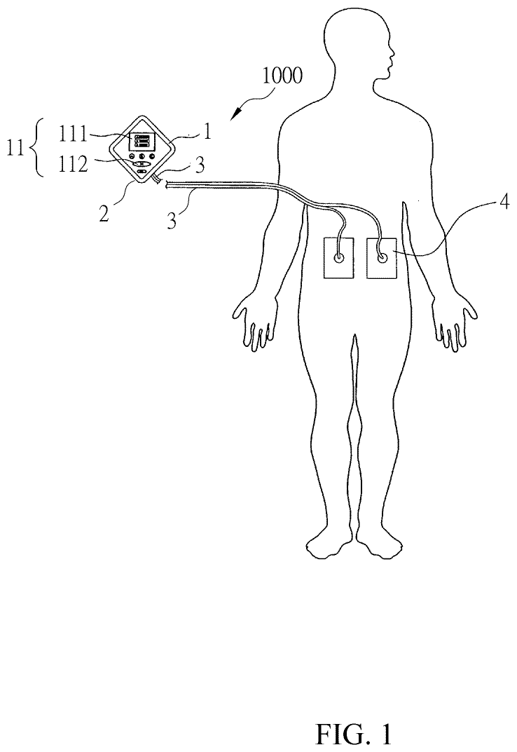

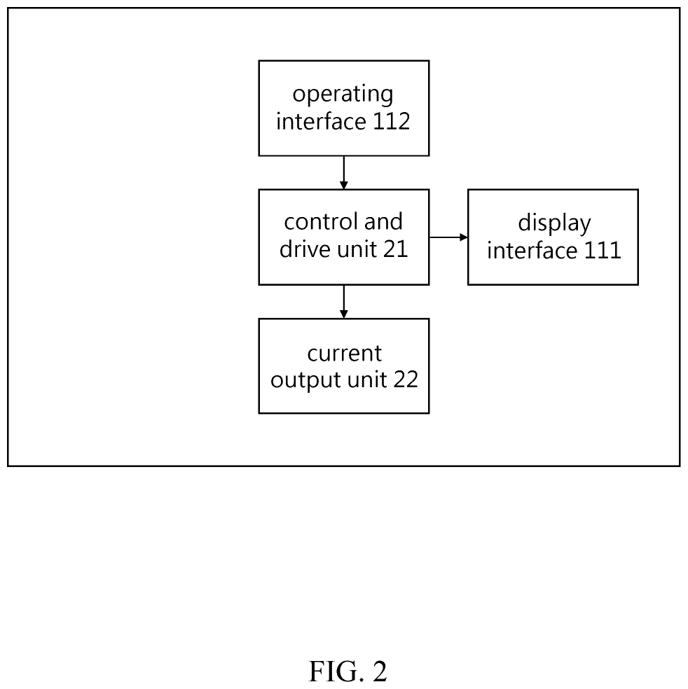

First, please refer to FIG. 1 and FIG. 2. FIG. 1 is a schematic view of an embodiment of the electrical stimulation generating device 1000 provided by the present invention. FIG. 2 is a schematic view of the system block inside the electrical stimulation generating device 1000 according to the present invention. As shown in FIG. 1, the electrical stimulation generating device 1000 includes a host machine 1, a signal generator 2, two signal transmission modules 3, and two electrode structures 4. The signal generator 2 is disposed in the host machine 1, and the two electrode structures 4 are placed on the abdominal area of an individual. The signal generator 2 generates and transmits a current to the individual through the electrode structures 4. It should be noted that in this embodiment, the electrical stimulation generating device 1000 uses two signal transmission modules 3 and two corresponding electrode structures 4 for illustration purposes. The configuration can be adjusted according to different use cases or requirements and is not limited to this embodiment.

In this embodiment, the host machine 1 includes an interface group 11, which is disposed on the surface of the host machine 1. The two ends of the signal transmission modules 3 are respectively connected between the signal generator 2 and the electrode structures 4. Specifically, the interface group 11 has a display interface 111 and an operating interface 112. The display interface 111 primarily provides users with a display screen for adjusting operations or displaying functional operations. The operating interface 112 is mainly used for users to select operational switches and command signals for preset program arrangements. It should be noted that in this embodiment, the display interface 111 and the operating interface 112 have their own independent functions. Considering the operational practicality and convenience of the electrical stimulation generating device 1000, these interfaces can also be combined into a single interface that performs both display and operational functions simultaneously, without limitation here.

Reference is next made to FIG. 2 for illustrating the signal generator 2. The signal generator 2 includes a control and drive unit 21 and a current output unit 22 for generating currents. These currents produce electrical stimulation pulses with frequencies ranging from 1 to 30 Hertz. The control and drive unit 21 can receive the triggers from command signals generated by user operating the operating interface 112 of the host 1, and then output the corresponding driving signal to control the current output unit 22. This allows the current output unit 22 to further adjust the output parameters of the electrical stimulation pulses, including their duty cycle, frequency, and potential difference, as pre-set by the program. Additionally, the control and drive unit 21 can control the powering on or off of the current output unit 22. On the other hand, the operating interface 112 can also transmit the selected parameter settings and timing information for output through the control and drive unit 21 to the display interface 111, allowing users to confirm the current device status. It should be noted that the control and drive unit 21 can be a combination of a controller and a drive circuit, and the aforementioned control and drive unit 21 is one as an example. The combination of the control and drive unit can be adjusted according to actual requirements and is not limited thereto.

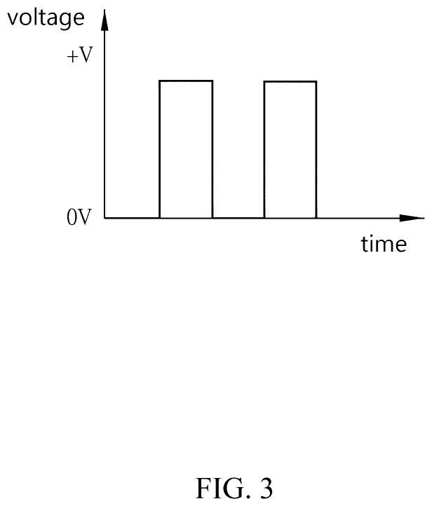

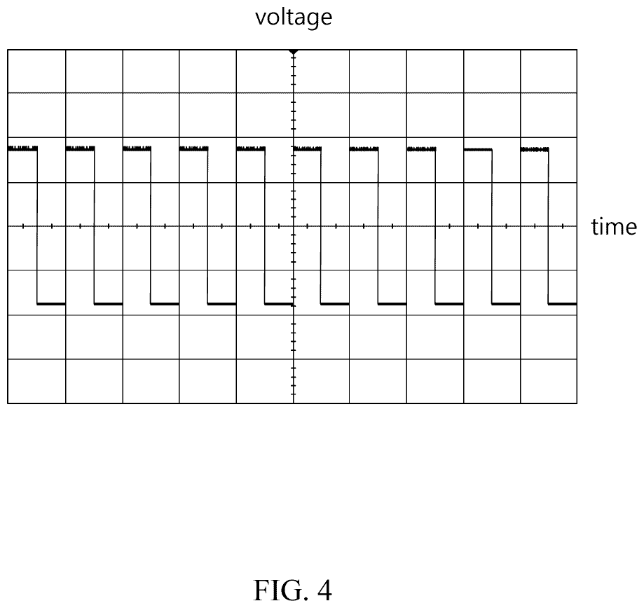

Next, one end of the signal transmission module 3 electrically connects to the signal generator 2, and the other end of the signal transmission module 3 electrically connects to one side of the electrode structure 4. The opposite side of the electrode structure 4 is used to contact the skin of the individual. The signal transmission module 3 is connected between the signal generator 2 and the electrode structure 4. When electrically connected, the signal generator 2 generates a current with an electrical stimulation pulse. Please refer to FIG. 3 to FIG. 4. As shown in FIG. 3, the electrical stimulation pulse is a single-phase pulse wave with a 50% duty cycle, and as shown in FIG. 4, it is presented as a schematic diagram in the oscilloscope with a 50% duty cycle. A 50% duty cycle represents a time ratio of 1:1 between the active time (when the electrical signals are output) and the inactive time (when the electrical signals are not output) of the single-phase pulse wave. In detail, the single-phase pulse wave is a fixed electrical pulse output in the positive phase, and during the working period, at least 75% of the pulses have a duty cycle of 50%+10%, which enables the electrical stimulation generating device 1000 to effectively and stably output signals.

In this embodiment, as shown in FIG. 4, the schematic view of the electrical stimulation pulses displayed on the oscilloscope illustrates that the electrical stimulation pulses are output as square waves in the positive phase. It should be noted that users can adjust the output of the controller and drive circuits of the control and drive unit 21 according to their needs. and select other single waveforms such as sine waves, triangular waves, or sawtooth waves that have similar effects to square waves, without limited thereto.

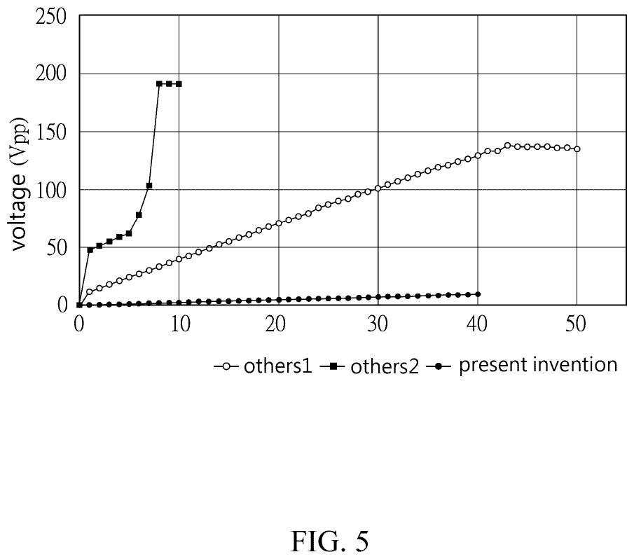

Specifically, please refer to FIGS. 5 to 7. The potential difference generated by the electrical stimulation pulses is less than 10 Vpp. The potential difference used in this invention is a voltage potential difference. Similarly, the potential difference used in this invention can be commonly found in the field, including peak-to-peak voltage (Vpp), maximum voltage (Vmax), or root mean square voltage (Vrms). The preferred range for the potential difference is less than 9.6 Vpp, and more preferably between 4.8 to 9.6 Vpp. Compared to commonly available electrical stimulation devices on the market, which typically have higher voltage outputs and less stable output amplitudes, the electrical stimulation device 1000 of this invention can stably output the voltage potential difference within the aforementioned range, providing users with a voltage potential difference suitable for application to the human body.

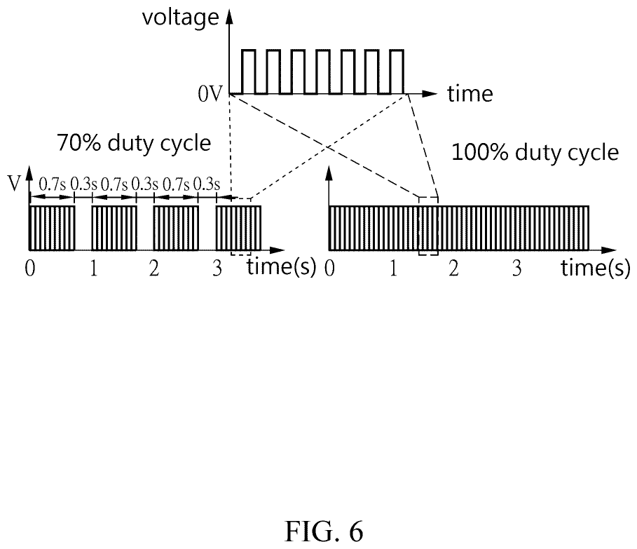

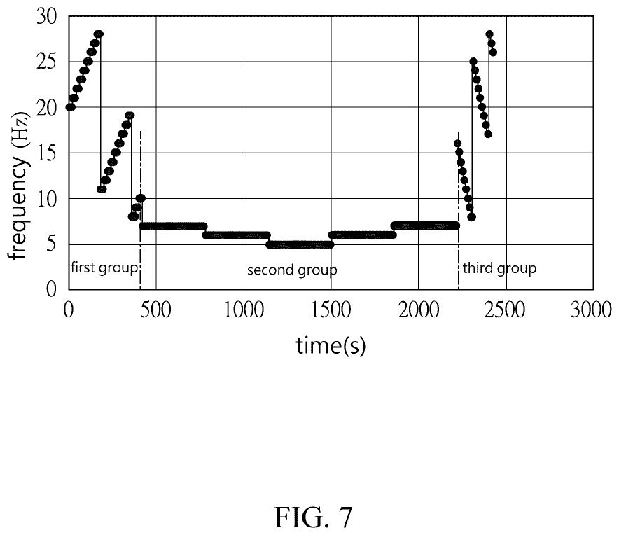

In this embodiment, as shown in FIGS. 6 to 7, the electrical stimulation pulses include a gradually increasing frequency group, a constant frequency group, or a gradually decreasing frequency group. Each of the gradually increasing frequency group, the constant frequency group, and the gradually decreasing frequency group is composed of a plurality of single-phase pulse groups, with the duty cycles of the single-phase pulse groups being more than 60%. In this embodiment, the duty cycles of the single-phase pulse groups are either 70% or 100%. A 70% duty cycle means that the single-phase pulse group outputs power for 0.7 seconds and rests for 0.3 seconds within each one second output cycle. In another embodiment, the single-phase pulse group has a 100% duty cycle, meaning that the single-phase pulse group continuously outputs power over time.

Specifically, as shown in FIGS. 6 to 7, the frequency used in this invention is an oscillation frequency generated by the signal generator 2. Similarly, the aforementioned oscillation frequency can also be replaced by commonly used vibration frequencies in the field. In this embodiment, the preferred frequency range for the gradually increasing frequency group and the gradually decreasing frequency group is 5 to 30 Hz, more preferably 8 to 28 Hz, and the preferred frequency range for the constant frequency group is 4 to 8 Hz, more preferably 5 to 7 Hz. The gradually increasing frequency group is composed of several frequencies, with each frequency in the group having an output time of 10 to 30 seconds. When a frequency ends, the next frequency in the group is executed, which is higher than the previous output frequency. The constant frequency group is composed of several frequencies, with each frequency in the group having an output time of 300 to 420 seconds. When a frequency ends, the next frequency in the group is executed. The gradually decreasing frequency group is composed of several frequencies, with each frequency in the group having an output time of 5 to 15 seconds. When a frequency ends, the next frequency in the group is lower than the previous output frequency. Each frequency group mentioned above has its respective output time. For example, the gradually increasing frequency group has an output time of 350 to 470 seconds, the gradually decreasing frequency group has an output time of 170 to 250 seconds, and the constant frequency group has an output time of 1750 to 1850 seconds. In this embodiment, as shown in FIG. 7, the electrical stimulation pulses generated by the electrical stimulation generating device 1000 are sequentially composed of a first group of gradually increasing frequency group, a second group of constant frequency group, and a third group of gradually decreasing frequency group. Each single-phase pulse wave has a duty cycle of 50%, and each single-phase pulse group has a duty cycle of 100%. The first group of gradually increasing frequency group sequentially includes a first gradually increasing frequency, a second gradually increasing frequency, and a third gradually increasing frequency. Next, the second group of constant frequency group sequentially includes a first constant frequency, a second constant frequency, a third constant frequency, a fourth constant frequency, and a fifth constant frequency. Finally, the third group of gradually decreasing frequency group sequentially includes a first gradually decreasing frequency, a second gradually decreasing frequency, and a third gradually decreasing frequency. Additionally, the first gradually increasing frequency to the third gradually increasing frequency in the gradually increasing frequency group, the first constant frequency to the fifth constant frequency in the constant frequency group, and the first gradually decreasing frequency to the third gradually decreasing frequency in the gradually decreasing frequency group each have their respective frequency ranges and output times. Specifically, the gradually increasing frequency group sequentially includes a first gradually increasing frequency with a frequency range of 15 to 30 Hz, a second gradually increasing frequency with a frequency range of 10 to 20 Hz, and a third gradually increasing frequency with a frequency range of 5 to 15 Hz. The constant frequency group includes a first constant frequency with a frequency range of 6 to 8 Hz, a second constant frequency with a frequency range of 5 to 7 Hz, a third constant frequency with a frequency range of 4 to 6 Hz, a fourth constant frequency with a frequency range of 5 to 7 Hz, and a fifth constant frequency with a frequency range of 6 to 8 Hz. The gradually decreasing frequency group sequentially includes a first gradually decreasing frequency with a frequency range of 20 to 5 Hz, a second gradually decreasing frequency with a frequency range of 30 to 15 Hz, and a third gradually decreasing frequency with a frequency range of 30 to 25 Hz. The output times for the first gradually increasing frequency and the second gradually increasing frequency are 150 to 200 seconds, and the output time for the third gradually increasing frequency is 50 to 70 seconds. The output times for the first constant frequency, the second constant frequency, the third constant frequency, the fourth constant frequency, and the fifth constant frequency are 350 to 370 seconds respectively. Finally, the output times for the first gradually decreasing frequency and second gradually decreasing frequency are 80 to 100 seconds, and the output time for the third gradually decreasing frequency is 10 to 50 seconds. The combination sequence of the gradually increasing frequency group, the constant frequency group, and the gradually decreasing frequency group, as well as the frequency range and the output time of the frequencies used in each group, can be adjusted according to actual needs without limitation thereto.

In this embodiment, the preferred frequency range of the electrical stimulation pulses is 1 to 30 Hz, more preferably 5 to 28 Hz. The frequency ranges used in the embodiments of this invention are 5 to 7 Hz and 8 to 28 Hz. These frequency ranges can be adjusted according to actual needs without limitation thereto.

Specifically, in this embodiment, the electrical stimulation generating device 1000 includes two signal transmission modules 3 for transmitting current, which are two signal transmission wires. The signal transmission module 3 connected to one end of the signal generator 2 includes corresponding positive and negative electrodes. At this point, the two signal transmission modules 3 connected to one end of the signal generator 2 can be combined into a single signal transmission module 3. When the aforementioned signal transmission module 3 is ready to connect to the electrode structure 4 and contact the living body, it can be divided into one signal transmission module 3 for the positive electrode and one for the negative electrode (as shown in FIG. 1). The quantity can be adjusted according to actual needs and is not limited thereto.

In this embodiment, the electrode structure 4 is a sheet-like, cloth-like or wearable device structure. It can be adhered to or in contact with a living body to transmit the current generated by the signal generator 2 into the body. The structure is not limited in the present invention. Moreover, in this embodiment, the electrode structure 4 has a contact surface, and the contact position of the contact surface is the skin of the living body. In this embodiment of the present invention, the preferred application position is the abdominal skin of the individual. This differs from typical electrical stimulation devices that are used near the head to stimulate specific nerves for therapeutic purposes. In this embodiment, the main application is to provide electrical stimulation pulses to the abdominal position of the individual, which can reduce unnecessary stimulation to the brain and potential damage. On the other hand, it can achieve the effect of improving sleep disorders through the interaction of electrical stimulation pulses with the living body.

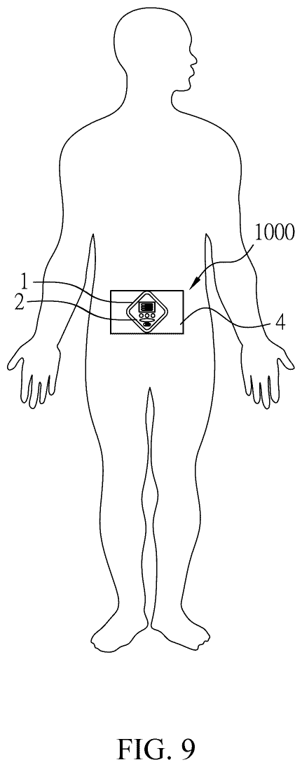

Please refer to FIGS. 9 to 12. In another embodiment of the present invention, the signal transmission module 3 is a combination of a signal transmission wire and magnetic clasp. Specifically, the signal generator 2 of the electrical stimulation generating device 1000 is installed in the host machine 1. The signal transmission wire of the signal transmission module 3 is also installed in the host machine 1 (not shown in the figure), with one end electrically connected to the signal generator 2 and the other end connected to a pair of magnetic clasps (female) on the surface of the host machine 1. When electrically connected, the current output from the signal generator 2 is transmitted through the signal transmission wire and the pair of magnetic clasps (female) on the surface of the host machine 1 for output.

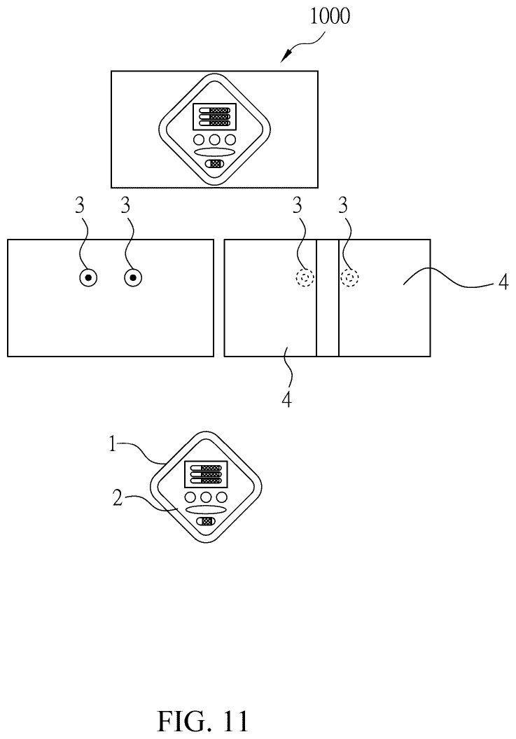

In other words, the signal transmission module 3 of the electrical stimulation generating device 1000 is designed with a signal transmission wire connected to magnetic clasps. The electrode structure 4 is a conductive carbon film combined with a hydrogel patch, and the quantity can be adjusted according to actual needs without limitation. In this embodiment, magnetic clasp type hydrogel patch is used to connect the host machine 1 to the individual. The surface of the hydrogel patch features an insulating layer and a pair of magnetic clasps (male) corresponding to the magnetic clasps (female) on the host machine 1. The magnetic clasps (male) penetrate the insulating layer and directly connect to the conductive carbon film underneath. Beneath the conductive carbon film is a layer of conductive hydrogel, which adheres to the individual's skin through the hydrogel patch. The pair of magnetic clasps (female) on the host machine 1 and the pair of magnetic clasps (male) on the patch are directly connected magnetically and transmit the electrical signals to the patch. The conductive carbon film evenly distributes the electrical signals transmitted by the connected magnetic clasps, and then transmits them through the hydrogel to the individual's epidermis, as shown in FIGS. 9 and 11. It should be noted that the hydrogel patches can be used in pairs to connect to the host machine 1 simultaneously. The two hydrogel patches can be used independently or connected as a single unit through an insulating layer. In this configuration, the two magnetic clasps are adjacent to the conductive carbon film/hydrogel but are not in contact with each other. Therefore, in this embodiment, the electrical stimulation generating device 1000 transmits the current to the individual in a wireless manner through the electrical signal transmission.

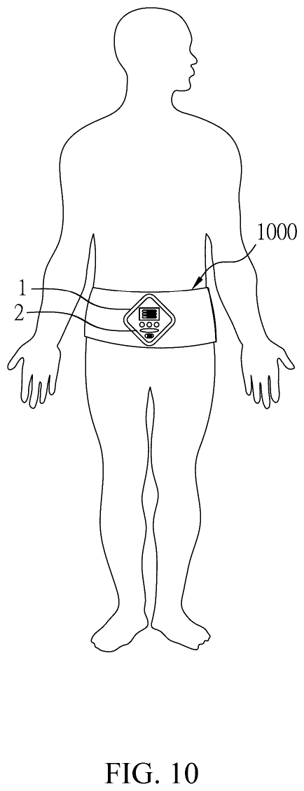

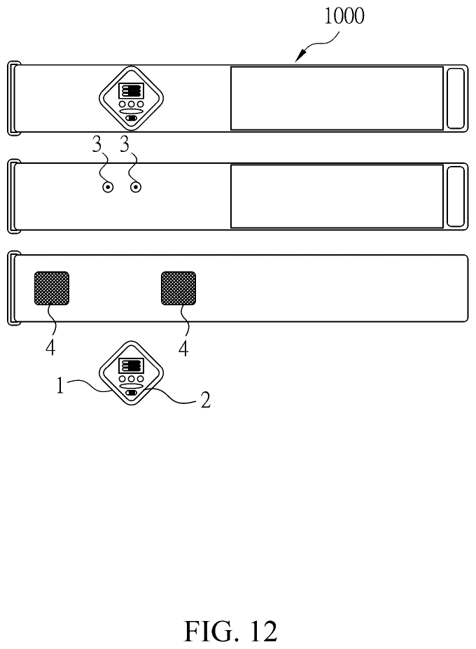

In another embodiment of the present invention, as shown in FIGS. 10 and 12, the signal transmission module 3 of the electrical stimulation generating device 1000 is also a combination of a signal transmission wire and magnetic clasp. The electrode structure 4 includes two carbon film electrodes, the number of which can be adjusted according to actual needs and is not limited thereto. In this embodiment, a conductive belt is used to connect the host machine 1 and the individual. The conductive belt is made of fabric material, with a pair of magnetic clasps (male) on the outer surface, and a pair of carbon film electrodes on the inner surface for contact with the individual's skin. The magnetic clasps and the carbon film electrodes are connected through signal transmission wires embedded within the fabric of the belt (not shown in the figure). The conductive belt has a plastic belt loop at one end and a hook and loop fastener on the other end. When wearing it, the magnetic clasps face outward, and the carbon film electrodes face inward. It wraps around the waist of the individual, with the hook and loop fastener end passing through the belt loop at the other end, folded back to adjust the appropriate tightness, and then secured with the hook and loop fastener. Additionally, the host machine 1 has a pair of magnetic clasps (female) that magnetically connect to the pair of magnetic clasps (male) on the outer side of the belt and transmit the electrical signals. The electrical signals are transmitted through signal transmission wires inside the fabric of the belt to individual carbon film electrodes, which then transmit the electrical signals into the individual through contact between the carbon film electrodes and the skin. Therefore, in this embodiment, the electrical stimulation generating device 1000 transmits the current to the individual in a wireless manner through the electrical signal transmission.

In this embodiment, the electrical stimulation generating device 1000 can be used to improve sleep disorders, including but not limited to insomnia, such as narcolepsy, sleep apnea, and other sleep abnormalities, REM sleep behavior disorder (RBD), bruxism, sleepwalking, and other parasomnias, sleep disorders related to mental, neurological, and other health issues, and snoring.

In this embodiment, a method is also provided for use with the aforementioned electrical stimulation generating device. This method includes generating an electrical stimulation pulse that can improve sleep disorders.

Following the above, the application method provides an electrical stimulation generating device 1000, which includes a host machine 1, a signal generator 2, two signal transmission modules 3, and two electrode structures 4. The signal generator 2 is disposed in the host machine 1. One end of the two signal transmission modules 3 is electrically connected to the signal generator 2, and the two electrode structures 4 are electrically connected to the other end of the signal transmission modules 3. The signal generator 2 generates a current that is transmitted to the individual through the electrode structures 4. The current generated by the signal generator 2 includes an electrical stimulation pulse with a frequency ranging from 1 to 30 Hertz, and includes a gradually increasing frequency group, a constant frequency group, or a gradually decreasing frequency group. The gradually increasing frequency group, the constant frequency group, and the gradually decreasing frequency group are each composed of a plurality of single-phase pulse groups, and a duty cycle of the single-phase pulse group is more than 60%. It should be noted that the use of two signal transmission modules 3 and corresponding connected electrode structures 4 in this embodiment serves as an illustrative example. The quantity and configuration can be adjusted according to the usage scenarios or needs of the electrical stimulation generating device 1000 in different embodiments, and are not limited to this embodiment.

### Embodiment 1

### [Improving Sleep Quality and Sleep Disorder]

To First, conduct experimental design and execution. Recruit two batches of participants, each consisting of five individuals, with a gender ratio ranging from 1:1 to 1:2. Provide each participant with an electrical stimulation generating device 1000 of the present invention. The method involves attaching the electrode structures 4 of the electrical stimulation generating device 1000 to the participant's abdominal area during sleep. Use it continuously for two weeks and record the usage time. Participants undergo the first sleep-related physiological monitoring and basic body parameter measurements the day before using the electrical stimulation generating device for therapy. Then, one day after the end of the two-week treatment period, participants undergo the second sleep-related physiological monitoring and basic body parameter measurements. Participants are required to complete the Pittsburgh Sleep Quality Index (PSQI) and the Insomnia Severity Index (ISI) before and after treatment. After the two-week treatment period, compare the improvements in PSQI and ISI.

The Pittsburgh Sleep Quality Index (PSQI) was developed by Dr. Buysse at the University of Pittsburgh in 1989. It is designed to collect objective data on participants' sleep over the past month. Based on the participants' sleep patterns, appropriate self-assessment questions are selected for evaluation. The scale includes nineteen independent items, categorized into seven components: subjective sleep quality, sleep latency, sleep duration, habitual sleep efficiency, sleep disturbance, use of sleep medication, and daytime dysfunction. Each of these seven components is rated on a scale from 0 to 3. The scores from these components are then summed to yield a total score ranging from 0 to 21 points. A higher score indicates poorer sleep quality, and a lower score suggests healthier sleep quality.

The Insomnia Severity Index (ISI) is an assessment tool developed by Dr. Charles M. Morin in 2011, widely used internationally to identify and classify the severity of insomnia. It contains five indicators of sleep-related problems, further divided into seven items with scores ranging from 0 to 4. The scores from these 7 items are then summed up, with a total score ranging from 0 to 28 points. A higher score indicates more severe insomnia, while a lower score suggests better sleep quality.

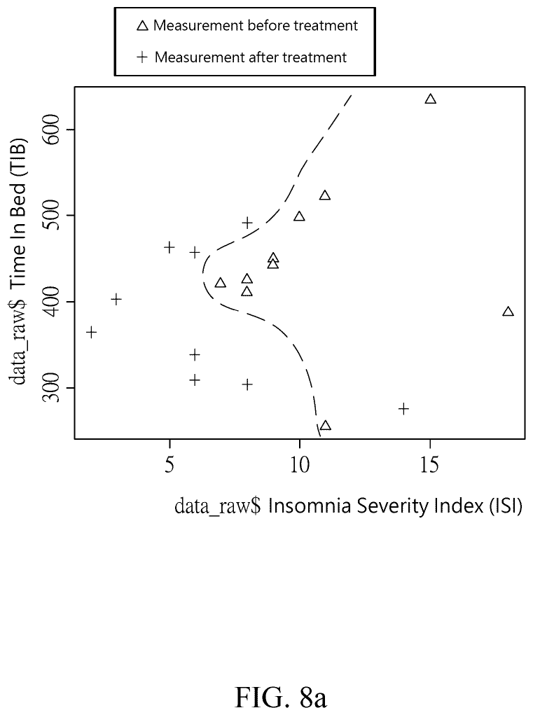

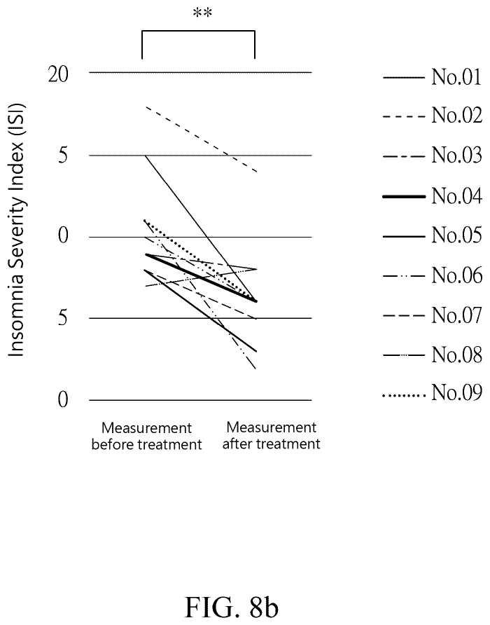

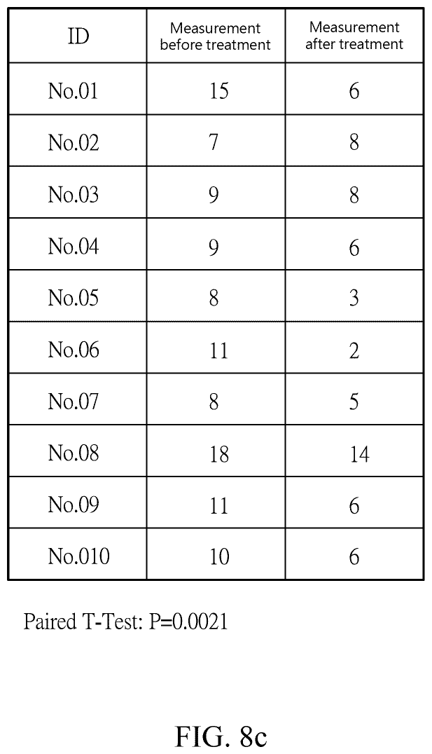

Please refer to FIGS. 8a to 8c for the experimental results of the Insomnia Severity Index (ISI) following the electrical stimulation therapy using the electrical stimulation generating device 1000 of the present invention. After the participants received the electrical stimulation pulses generated by the electrical stimulation generating device 1000 of the present invention for two weeks, the experimental data were obtained and statistically analyzed using the t-test.

The above experimental results demonstrate that compared to before electrical stimulation therapy, there was a significant improvement in the Insomnia Severity Index (ISI), as shown in FIGS. 8a and 8b. After treatment with the electrical stimulation generating device 1000, the severity of insomnia was significantly reduced (P<0.01), accompanied by a decrease in the time in bed (TIB) as the severity of insomnia decreased. On the other hand, as shown in FIG. 8c, evaluations from the ISI scores indicated significant decreases in scores for nearly all participants (P<0.01). These results indicate that participants using the electrical stimulation generating device 1000 of the present invention, which generates the electrical stimulation pulses including the single-phase pulses with at least 60% duty cycle formed by the single-phase pulses with the duty cycle of 50%, along with the frequencies, and the potential differences, which are then transmitted to the individuals, experienced a significant reduction in the severity of their insomnia. This device effectively improved both their sleep quality and sleep disorders.

Although the present invention has been described in considerable detail regarding certain embodiments thereof, other embodiments are possible. Therefore, the spirit and scope of the appended claims should not be limited to the description of the embodiments contained herein.

1. An electrical stimulation generating device for improving sleep disorders, comprising:
a host machine;
a signal generator being disposed within the host machine for generating a current;
at least one signal transmission module, wherein one end of which is electrically connected to the signal generator;
at least one electrode structure being electrically connected to the other end of the signal transmission module, and having a contact surface for contacting the skin of an individual and being transmitting the current to the individual;
wherein the current generated by the signal generator has an electrical stimulation pulse with a frequency of 1 to 30 Hz, and includes a gradually increasing frequency group, a constant frequency group, or a gradually decreasing frequency group, the gradually increasing frequency group, the constant frequency group, and the gradually decreasing frequency group are each composed of a plurality of single-phase pulse groups, and a duty cycle of the single-phase pulse group is more than 60%.

2. The electrical stimulation generating device for improving sleep disorders as claimed in claim 1, wherein the frequency of the gradually increasing frequency group and the gradually decreasing frequency group range from 5 to 30 Hz, and the frequency of the constant frequency group ranges from 4 to 8 Hz.

3. The electrical stimulation generating device for improving sleep disorders as claimed in claim 1, wherein the output time of the gradually increasing frequency group is between 350 and 470 seconds, the output time of the gradually decreasing frequency group is between 170 and 250 seconds, and the output time of the constant frequency group is between 1750 and 1850 seconds.

4. The electrical stimulation generating device for improving sleep disorders as claimed in claim 1, wherein the gradually increasing frequency group sequentially includes a first gradually increasing frequency, a second gradually increasing frequency, and a third gradually increasing frequency, the constant frequency group sequentially includes a first constant frequency, a second constant frequency, a third constant frequency, a fourth constant frequency, and a fifth constant frequency, and the gradually decreasing frequency group sequentially includes a first gradually decreasing frequency, a second gradually decreasing frequency, and a third gradually decreasing frequency.

5. The electrical stimulation generating device for improving sleep disorders as claimed in claim 4, wherein each of the first gradually increasing frequency to the third gradually increasing frequency, the first constant frequency to the fifth constant frequency, and the first gradually decreasing frequency to the third gradually decreasing frequency have their respective frequency ranges and output times.

6. The electrical stimulation generating device for improving sleep disorders as claimed in claim 1, wherein the duty cycles of the single-phase pulse groups are 70% or 100%.

7. The electrical stimulation generating device for improving sleep disorders as claimed in claim 1, wherein the single-phase pulse groups are composed of a plurality of single-phase pulses with the duty cycle of 40% to 60%.

8. The electrical stimulation generating device for improving sleep disorders as claimed in claim 1, wherein the waveform of the electrical stimulation pulse is a square wave, a sine wave, a triangle wave, or a sawtooth wave.

9. The electrical stimulation generating device for improving sleep disorders as claimed in claim 1, wherein a potential difference of the current is less than 10 Vpp.

10. The electrical stimulation generating device for improving sleep disorders as claimed in claim 1, wherein the signal transmission module is a signal transmission wire or a combination of the signal transmission wire and a magnetic clasp.

11. The electrical stimulation generating device for improving sleep disorders as claimed in claim 1, wherein the electrode structure contacts the abdominal skin of the individual.

12. A method for applying to the electrical stimulation generating device for improving sleep disorders as claim 1.
